(19) United States
(12) Patent Application Publication
(10) Pub. No.: US 2024/0150720 A1
(43) Pub. Date: May 9, 2024

(54) INFECTED CELL CULTURES

(71) Applicant: Merck Patent GmbH, Darmstadt (DE)

(72) Inventors: Thomas SPANGENBERG, Geneva (CH); Beatrice Greco, Ivrea (TO) (IT); Paula Maria Marques Leal Sanches Alves, Oeiras (PT); Manuel José Teixeira Carrondo, Caxias (PT); Ana Catarina Mauricio Brito Ataíde, Amadora (PT); Sofia Raquel Paulo Rebelo, Caldas da Rainha (PT); Francisca Maria De Andrade Terras Arez, Odivelas (PT); Daniel Filipe Mestre Simão, Faro (PT); Rui Miguel Prudêncio Pignatelli, Lisboa (PT); Diana Marisa Pinto Freire Fontinha, Odivelas (PT); Marta Monteiro Maia Machado, Odivelas (PT)

(73) Assignee: Merck Patent GmbH, Darmstadt (DE)

(21) Appl. No.: 18/414,253

(22) Filed: Jan. 16, 2024

Related U.S. Application Data
(62) Division of application No. 16/492,071, filed on Sep. 6, 2019.

(30) Foreign Application Priority Data
Mar. 10, 2017 (EP) 17160277.4

(51) Int. Cl. C12N 5/071; G01N 33/50

(52) U.S. Cl. CPC C12N 5/0671; G01N 33/5067; C12N 2527/00; G01N 2333/445

(57) ABSTRACT

The present invention relates to 3D cell cultures, which contains hepatic cells and are infected by a pathogen, methods for preparing such cell cultures and uses thereof.

### BACKGROUND OF THE INVENTION

The present invention relates to 3D cell cultures, which contain hepatic cells and are infected by a pathogen, methods for preparing such cell cultures and uses thereof.

A variety of pathogens transit or mature in the liver, in the case of infections by Plasmodium parasites, the causative agents of malaria, when an infected mosquito takes a blood meal from a mammalian, including human beings, sporozoites present in the salivary glands of the mosquito are inoculated into capillaries of the upper dermis from where they will reach the portal circulation. Subsequently, they travel to the liver where they invade hepatic cells. Here, the parasites undergo a process termed exoerythrocytic parasite development, in which the hepatic parasites replicate asexually and differentiate into merozoites. Upon completion of this replicative phase, 10 000-40 000 merozoites are eventually released into the blood stream, at which point they invade and replicate inside erythrocytes, initiating a new cycle of asexual replication and growth [Prudêncio, M., Rodriguez, A., & Mota, M. M. (2006). The silent path to thousands of merozoites: the Plasmodium liver stage. Nature Reviews. Microbiology, 4(November), 849-56]. When a single parasite is present inside an erythrocyte, it is termed an early trophozoite. The trophozoite grows and then begins to asexually replicate, a phenomenon known as schizogony. When schizonts are sufficiently mature, the erythrocytes rupture, releasing merozoites with a subsequent increase in the number of circulating malaria parasites. During this phase of infection, some parasites differentiate into gametocytes, both male and female. These are then taken up by mosquitoes during a blood meal and transform into male and female gametes. The union of male and female gametes forms diploid zygotes, which in turn become ookinetes. These ookinetes migrate to the midgut of the insect, pass through the gut wall and form the oocysts in the haemolymph. Meiotic division of the oocysts occurs, leading to maturation and rupture to release sporozoites, which then migrates to the salivary glands of the female Anopheles mosquito ready to continue the cycle of transmission back to man [Douglas, N. M., Simpson. J. a., Phyo, A. P., et al. (2013) Gametocyte dynamics and the role of drugs in reducing the transmission potential of Plasmodium vivax. Journal of Infectious Diseases, 208, 801-812; Swann, J., Corey, V., Scherer, C. a., et al, (2016) High-Throughput Luciferase-Based Assay for the Discovery of Therapeutics That Prevent Malaria. ACS Infectious Diseases, acsinfecdis.5b00143]. All mammalian-infective Plasmodium species transit and mature through the liver but P. vivax and P. ovale can generate latent hepatic forms—known as hypnozoites—, which can lead to disease relapses. Primaquine is currently the only marketed monotherapy drug for the latter indication thought to exert its effect by metabolic activation. For liver stage prophylaxis, atovaquone is used in combination with another partner drug.

The study of the liver stage of Plasmodium's life cycle has greatly benefited from the use of hepatic cell lines (e.g. HepG2, Huh7, HC04) and primary cultures of human hepatocytes [Prud\tilde{e}ncio, M., Mota, M. M., & Mendes, A. M. (2011), A toolbox to study liver stage malaria. Trends in Parasitology]. These cells have been mostly explored in 2D culture systems and combined with Plasmodium strains that constitutively express a reporter gene, either green fluorescent protein (GFP) or Luciferase (Luc), to follow and address specific features of the parasite's hepatic development. For example, the requirement of sporozoite cell traversal process towards the effective invasion of the final hepatocyte [Mota, M. M., Hafala, J. C. R., & Rodrigues, A. (2002) Migration through host cells activates Plasmodium sporozoites for infection, Nat Med, (9(11), 548. Risco-Castillo. V., Topçu, S., Marinach, C. et al. (2015) Malaria sporozoites traverse host cells within transient vacuoles. Cell Host and Microbe. 18, 593-603], development throughout liver infection inside transient vacuoles [Risco-Castillo, V., Topçu, S., Marinach, C. et al. (2015) Malaria sporozoites traverse host cells within transient vacuoles. Cell Host and Microbe, 18, 593-603] and the role and specific localization of key proteins involved in disrupting the parasitophorous vacuole during late liver-stage infection [Burda. P. C., Roeill, M. a., Schaffner, M., et at (2015) A Plasmodium phospholipase is involved in disruption of the liver stage parasitophorous vacuole membrane. PLoS Pathogens, 11, e1004760] have been addressed taking advantage of such models. In vitro hepatic models that allow P. falciparum and P. vivax development, the two most clinically relevant human-infective Plasmodium species, have also been developed [Chattopadhyay, R., Velmurugan, S., Chakiath, C., et al. (2010) Establishment of an in vitro Assay for Assessing the Effects of Drugs on the Liver Stages of Plasmodium vivax Malaria. PLoS ONE, 5(12), 1-8; Dumoulin. P. C., Trop, S. A., Ma, J., et al, (2015) Flow cytometry based detection and isolation of Plasmodium falciparum liver stages in vitro. PLoS ONE, 10, 1-2; March, S., Ng, S., Velmurugan, S., et at (2013) A microscale human liver platform that supports the hepatic stages of Plasmodium falciparum and vivax. Cell Host and Microbe. 14(1), 104-115]. The relevance of the liver microenvironment's physicochemical features for Plasmodium infection and development has been shown in vitro by co-culturing primary hepatocytes and stromal cells. More specifically, hypoxia has been demonstrated to enhance the development of different Plasmodium species [Ng, S., March, S., Galstian, A., Hanson. K., et al. (2014) Hypoxia promotes liver stage malaria infection in primary human hepatocytes in vitro, 215-224]. Furthermore, these in vitro hepatic models have contributed to the development of high-throughput screening platforms, which can aid in the identification and development of anti-Plasmodium agents [Derbyshire. E. R., Prudbncio. M., Mota, M. M., et al. (2012) Liver-stage malaria parasites vulnerable to diverse chemical scaffolds. Proceedings of the National Academy of Sciences of the United States of America, 109(22), 8511-6; Swann, J., Corey, V., Scherer, C. A., et al. (2016) High-Throughput Luciferase-Based Assay for the Discovery of Therapeutics That Prevent Malaria. ACS Infectious Diseases, 2(4): 281-293.].

The previously established in vitro models of Plasmodium infection have proved their importance towards an increased understanding of the specific features of parasite biology. Nevertheless, few models and assays have addressed the Liver dormant forms of the parasite (hypnozoite), due not only to the difficulties inherent to obtaining P. vivax sporozoites to be used experimentally, but also to the lack of hepatic cell models that can be maintained in culture for long time periods with high viability and function. The available reports described in the literature are based on 2D cultures of primate primary hepatocytes infected with P. cynomoigi [Dembele, L., Gego, A., Zeeman, A. M., et al. (2011) Towards an in vitro model of plasmodium hypnozoites suitable for drug discovery. PLoS ONE, 6(3), 1-7; Voorberg-van der Wel, A., Zeeman, A. M., van Amsterdam, S. M., et al. (2013) Transgenic Fluorescent Plasmodium cynomolgi Liver Stages Enable Live Imaging and Purification of Malaria Hypnozoite-Forms. PLoS ONE, 8(1)], where hypnozoites are distinguished from normal developing schizonts by their resistance to atovaquone. However, due to the limited time of primary hepatocytes cultures (up to two weeks when in collagen sandwich cultures), the reactivation capacity of hypnozoites was not assessed. One recent report contemplating an improved collagen sandwich system was able to maintain a co-culture of primary hepatocytes and a hepatoma cell line for 40 days after P. cynomolgi infection [Dembélé, L. Franetich, J., Lorthiois, A., et al. (2014) Persistence and activation of malaria hypnozoites in long-term primary hepatocyte cultures. Nature Medicine, 20(3), 307-312]. With this model, the authors demonstrated the persistence of hypnozoites for more than one month in the hepatocytes and showed their activation towards normal development beyond three weeks after infection. However, this model's technical complexity decreases its throughput, compromising the applicability in drug screening settings.

Recent developments in human hepatic 3D cell models have demonstrated the ability to recapitulate many important hepatocyte features in stirred-tank bioreactors (STB), by generating cell spheroids of hepatic cell lines or freshly isolated human hepatocytes (Rebelo, S. P., Costa, R., Estrada, M., et al. (2014) HepaRG microencapsulated spheroids in DMSO-free culture: novel culturing approaches for enhanced xenobiotic and biosynthetic metabolism. Arch Toxicol; Tostões, R. M., Leite, S. B., Serra, M., et al. (2012) Human liver cell spheroids in extended perfusion bioreactor culture for repeated-dose drug testing. Hepatology, 55(4), 1227-1236; Rebelo, S., Costa, R., Sousa, M. F. O., at al. (2015) Establishing Liver Bioreactors for In Vitro Research. Protocols in In Vitro Hepatocyte Research, 1250, 1-390). Importantly, physiochemical parameters such as oxygen and pH, as well as the feeding regimen can be controlled in the STB, allowing the reproducibility and stability of hepatic phenotype in long-term cultures, as well as modeling specific characteristics of the liver (e.g, physiological periportal or perivenous oxygen concentrations). Moreover, the scalability of STB enables the production of large quantities of hepatic spheroids that can be used to feed high-throughput screening platforms [Rebelo, S. P., Costa, R. Estrada, M., et al. (2014) HepaRG microencapsulated spheroids in DMSO-free culture: novel culturing approaches for enhanced xenobiotic and biosynthetic metabolism, Arch Toxicol; Tostdes, R. M., Leite, S. B., Serra, M., et at (2012) Human liver cell spheroids in extended perfusion bioreactor culture for repeated-dose drug testing. Hepatology, 55(4), 1227-1236; Rebelo, S., Costa, R., Sousa, M. F. O., et at (2015) Establishing Liver Bioreactors for In Vitro Research. Protocols in In Vitro Hepatocyte Research, 1250, 1-390].

### DESCRIPTION OF THE INVENTION

The present invention provides a 3D cell culture comprising cell aggregates, which contain hepatic cells, wherein the cell aggregates are infected by a pathogen.

The 3D cultures of the present invention have a good long-term stability and are therefore useful for drug screening and vaccine development.

In a particular embodiment of the present invention, the pathogen is a parasite.

In another specific embodiment of the present invention, the 3D cell culture is a mono-culture or a co-culture.

In another specific embodiment of the present invention, the hepatic cells are selected from a group of cell sources comprising primary human, murine and primate hepatocytes, cell lines such as HC-04, HepG2, HepaRG and/or Huh7, and hepatocyte-like cells derived from pluripotent or multipotent stem cells.

Yet, in a further specific embodiment of the present invention, the hepatic cells are selected from a group of cell lines comprising primary human and primate hepatocytes, HC-04, HepG2, HepaRG and/or Huh7.

In a specific embodiment, the 3D cell culture is a co-culture, which contains cells from at least one hepatic cell type (such as in particular primary human and primate hepatocytes, HC-04, HepG2, HepaRG and/or Huh7) and non-parenchymal cells such as endothelial, immune or stromal cells (Human Mesenchymal Stem Cells, macrophages, fibroblasts or stellate cells).

In a very specific embodiment, the 3D cell culture is a co-culture, which contains cells from at least one hepatic cell type (such as in particular primary human and primate hepatocytes, HC-04, HepG2, HepaRG and/or Huh7) and Human Mesenchymal Stem Cells.

In another particular embodiment, the 3D cell culture according to the invention contains cell aggregates having an average diameter in the range of 50 μm to 200 μm (by microscopy). The cell aggregates can be spheroids.

A further specific embodiment refers to a 3D cell culture, wherein the parasite is from the genus Plasmodium, preferably selected for a group comprising P. berghei, P. falciparum, P. vivax, P. ovale, P. cynomolgi, P. malariae and P. knowlesi. For infection of the cell aggregates, the sporozoites are put in contact with the cell aggregates.

In a further specific embodiment, the pathogen is a reporter strain such as e.g. a Plasmodium species expressing green fluorescent protein (GFP) or luciferase (Luc). Reporter strains allow a very easy detection and monitoring of the infection rate.

In another embodiment of the present invention, the 3D cell culture contains a cell culture medium, wherein culture medium is a mammalian cell culture medium (such as in particular DMEM supplemented or not with F12-supplement). In a preferred embodiment, the cell culture medium further contains up to 20% FBS concentration.

The choice of the mammalian culture medium depends on the cell line e.g. for HC-04 cell cultures DMEM supplemented with F12 is suitable, for HepG2 DMEM (without F12) is suitable. For HepaRG and primary hepatocytes the cell culture medium as described e.g in [Rebelo, S. P. et al, (2014). HepaRG microencapsulated spheroids in DMSO-free culture: novel culturing approaches for enhanced xenobiotic and biosynthetic metabolism. Arch Toxicol], [Tostdes, R. M., et at (2012). Human liver cell spheroids in extended perfusion bioreactor culture for repeated-dose drug testing. Hepatology, 55(4), 1227-1236], or [Rebelo, S., Costa, R., Sousa, M. F. Q., Brito, C., & Alves, P. M. (2015). Establishing Liver Bioreactors for In Vitro Research. Protocols in In Vitro Hepatocyte Research, 1250, 1-390] can be used.

Another embodiment of the invention relates to a 3D cell culture, wherein the 3D cell culture further contains soluble extracellular matrix (preferably laminin, fibronectin and/or collagen) and/or a biocompatible biomaterial (e.g. alginate, chitosan, polylactic acid).

Another embodiment of the invention relates to a 3D cell culture, wherein the 3D cell culture further contains soluble extracellular matrix, preferably laminin, fibronectin, and/or collagen.

In a specific embodiment of the present invention, the 3D cell culture is characterized by an infection rate of at least 0.01% (measured e.g. by fluorescence and luminescence; infection by parasites that do not express reporter genes can be assessed by a variety of methods, including immunofluorescence microscopy following staining with appropriate antibodies, and quantitative real-time PCR employing Plasmodium-specific primers and primers for appropriate housekeeping host genes [Prudencio, M., Mota, M. M., & Mendes. A. M. (2011). A toolbox to study liver stage malaria. Trends in Parasitology]).

Accordingly, a very specific embodiment refers to a 3D cell culture, wherein
the cell culture is infected by a pathogen, which is a parasite from the genus Plasmodium, preferably selected for a group comprising P. berghei, P. falciparum, P. vivax, P. ovale, P. cynomolgi, P. malariae and P. knowlesi;
the hepatic cells are selected from a group of sources comprising primary human, murine and primate hepatocytes, cell lines such as HC-04, HepG2, HepaRG and/or Huh7, and hepatocyte-like cells derived from pluripotent/multipotent stem cells.
the cell culture contains a mammalian cell culture medium (and preferably the culture medium further contains up to 20% FBS concentration).

Another very specific embodiment refers to a 3D cell culture, wherein
the cell culture is infected by a pathogen, which is a parasite from the genus Plasmodium, preferably selected for a group comprising P. berghei. P. falciparum, P. vivax, P. ovale, P. cynomolgi, P. malariae and P. knowlesi;
the hepatic cells are selected from a group of cell lines comprising primary human and primate hepatocytes, HC-04, HepG2, HepaRG and/or Huh7; and
the cell culture contains a mammalian cell culture medium (and preferably the culture medium further contains up to 20% FBS concentration).

Preferably, such a 3D cell culture contains cell aggregates having an average diameter in the range of 50 μm to 200 μm (the corresponding cell aggregates are useful for long-term cultures). Such a cell culture may further contain soluble extracellular matrix (preferably laminin, fibronectin and/or collagen) or a biocompatible biomaterial (e.g. alginate, chitosan, polylactic acid).

For the infection, sporozoites are put in contact with the 3D cell aggregates.

The above described infected 3D cell cultures according to the invention are useful e.g. for drug screening and vaccine development. In particular, the cell cultures according to the present invention have following advantages: an improved long-term stability (the infected 3D cell cultures can be cultured up to 2/3 months), good culture functionality, and/or an improved infectivity.

The present invention further provides a multi-well plate containing a 3D cell culture of hepatic cells as described above. Multi-well plates are useful e.g. for high throughput screenings in drug or vaccine development.

In addition, the invention provides a method for the production of a 3D cell culture containing hepatic cells, comprising following steps:
(step a) Inoculation of a single-cell suspension containing hepatic cells and/or other cell types (such as in particular Mesenchymal stem cells and/or non-parenchymal liver cells like e.g. Kupffer, stellate, endothelial cells) expanded in a 2D culture in an agitation-based culture system;
(step b) Agitation of the resulting cell culture at an agitation rate of 40 to 110 rpm; and
(step c) incubation of the resulting 3D cell culture containing cell aggregates (which preferably have an average diameter in the range of 50 μm to 200 μm) with a pathogen (wherein the cell-to-pathogen ratio is preferably between 10:1 and 1:5000).

The invention also provides a method for the production of a 3D cell culture containing hepatic cells, comprising following steps:
(step a) Inoculation of a single-cell suspension containing hepatic cells expanded in a 2D culture in an agitation-based culture system (step a));
(step b) Agitation of the resulting cell culture at an agitation rate of 40 to 90 rpm); and
(step c) Incubation of the resulting 3D cell culture containing cell aggregates (which preferably have an average diameter in the range of 50 μm to 200 μm) with a pathogen (wherein the cell-to-pathogen ratios is preferably between 5:1 and 1:5000).

The 2D culture used for the inoculation is obtainable by different well known procedures (see e.g. Freshney RI: Culture of Animal Cells. 6th Ed. Hoboken, NJ, USA: John Wiley & Sons, Inc., 2010).

In a preferred embodiment of the method described above, the concentration of the single-cells in the in the cell medium is in range of 0.1×10⁶ to 1×10⁶ cell/mL. Furthermore, the agitation-based culture system is preferably a stirred-tank bioreactor or a spinner vessel. Preferably, the inoculation is also performed in the agitation-based culture system.

In another preferred embodiment of the described method, the inoculation (step a) and/or agitation (step b) and/or incubation (step c) is performed at a temperature in the range of 37° C.±2° C. in humidified atmosphere (up to 95% of relative humidity). 5%-10% of CO₂ in air. In a specific embodiment, the agitation is performed for a time period of several weeks (for example 1-2 weeks) (wherein the culture medium is exchanged if needed, preferably every 2-3 days).

In another very important embodiment of this method, the 3D cell culture is centrifuged at up to 1800×g during the incubation (step c)). The centrifugation promotes cell-pathogen contact by increasing the local concentration of cells and pathogens in a certain layer within the culture medium due to their density. In this embodiment, the cell culture medium volume is preferably kept constant (which means that there is no or no significant reduction of the cell culture volume over time). Nevertheless, the culture medium can be exchanged if needed (preferably without changing the overall volume), preferably the culture medium is exchanged every 2-3 days. Most preferably, moderate acceleration and brake settings are used for this centrifugation procedure in order to avoid aggregate/spheroid fusion. According to the present invention, the conditions described above can be referred to as “static incubation conditions”.

Yet, in a specific embodiment of the present invention the cell culture is centrifuged at up to 1800×g during the incubation (step c)), wherein the cell culture volume is preferably kept at a constant level.

In another embodiment, the 3D cell culture is exposed to agitation during the incubation (step c)) (preferably in a spinner vessel or multiwell-plate). The agitation speed is preferably within a range of 110 to 40 rpm. Agitation is useful to promote cell-pathogen contact. Moreover, in such an embodiment the cell culture medium volume is preferably reduced during incubation to 10-75% of the starting volume. The reduction of the cell culture volume results in an increased concentration. This can further promote pathogen-cell contact. According to the present invention the conditions described above can be referred to as “dynamic conditions”. Under these conditions the incubation can e.g. be performed in approximately 2 h under continuous agitation, with tuning of the agitation speed within a range of 110 to 40 rpm). This is embodiment is particular suitable for large volume cell cultures and it can also be advantageous if the formation of large aggregates is desired.

Consequently, in a specific embodiment of the present invention the cell culture is exposed to agitation during the incubation (step c)), wherein agitation speed is preferably within a range of 110 to 40 rpm, and wherein the cell culture volume is preferably reduced to 10-75% of the starting volume.

In a further embodiment of the method for the production of a 3D cell culture according to the invention, the incubation is performed under static conditions, wherein 3D cell culture containing the cell aggregates together with the pathogen is exposed to centrifugation at up to 1800×g, or the incubation is performed under dynamic conditions, wherein the cell culture volume is reduced (preferably to 10-75% of the starting volume) and the cell culture is exposed to agitation (wherein agitation speed is preferably within a range of 110 to 40 rpm).

In another embodiment the cell culture medium volume is reduced during the infection to 50-75% of the starting volume under continuous agitation. This can be particular suitable if the formation of large aggregates is desired.

The present invention also relates to a 3D cell culture of hepatic cells obtainable with a method or the production of a 30 cell culture of hepatic cells as described above.

The invention also provides a screening method, comprising following steps:
Incubation of a 3D cell culture of hepatic cells with a compound, wherein the hepatic cell culture is a cell culture as described above or a cell culture obtained with a method described above;
Monitoring of pathogen invasion, compound clearance and/or development of host cells.

The monitoring can be performed using different well-known techniques (such as e.g. fluorescence, luminescence, immunofluorescence and antigen detection).

The invention further relates to a use of a 3D cell culture according to the invention to determine a cytotoxic effect and/or metabolic properties of a compound contacted with the 3D cell culture and/or an effect of a compound contacted with the cell culture on the pathogen (preferably for drug screening purposes).

The 3D cultures and methods according to the present invention are suitable for the screening of novel anti-infective compounds e.g. because of the mature phenotype of the hepatocytes that can be achieved, the high infectivity that can be achieved (e.g. infection rate of up to 3% achievable for P. berghei cells) and the ease of the pathogen reporter system. In addition, it is possible to unveil the compound action point on the infection process by incubation of the compound at specific time periods (see FIG. 1) The invention further relates to a use a 3D cell culture according to the invention for vaccine development.

The invention further relates to a screening assay for an anti-parasitic drug and/or a vaccine.

The invention also relates to a kit for the screening for a drug (preferably an anti-parasitic drug) and/or a vaccine comprising a 3D cell culture according to the invention.

The production methods according to the present invention allow to produce such 3D cell cultures in large quantities, which is very useful e.g. for high-throughput screening.

In the context of the present invention, the term ‘3D cell culture’ or ‘3D culture’ refers to a cell culture comprising three dimensional cell aggregates (including in particular spheroids). In 3D cultures, the cells are attached to one another, thus allowing cell-to-cell interactions.

The term ‘2D cell culture’ or ‘20 culture’ refers to a two dimensional cell culture.

The term ‘cell aggregate’ refers to a 3D cell aggregate (in particular spheroids).

The term ‘co-culture’ refers to an in vitro cell culture containing at least two distinct cell types, wherein at least cell type is a hepatic cell type. Accordingly, a co-culture may for example contain cells from two (or more) different hepatic cell types or a co-culture may contain cells from one (or more) hepatic cell type(s) in combination with cells from at least one (or more) further non-hepatic cell type(s). The term ‘mono-culture’ refers to an in vitro cell culture containing only one (hepatic) cell type.

In view of a cell aggregate, the term ‘infected’ (or ‘infected aggregate’) means that at least one cell per cell aggregate is infected. In the context of the present invention, the infected cell is a hepatic cell.

In the context of the present invention, “hepatocyte-like cells derived from pluripotent or multipotent stem cells”, are undifferentiated cells that have the potential to differentiate into hepatic cells. “Pluripotent stem ceils” can differentiate into 3 germ layers, while “multipotent stem cells” refer to hepatic progenitor cells, that can only differentiate into tissue-specific cell types.

The term “single-cell suspension” refers to a suspension of cells that, basically comprises individual, non-aggregated cells.

### BRIEF DESCRIPTION OF THE FIGURES

FIG. 1—Example of a possible compound incubation regime to be used on drug screening for anti-Plasmodium agents. (A) Incubation of the drug from 1 hour before the addition of the pathogens up to 2 or 7 days later; (B) Incubation of the drug for 1 hour before the addition of Plasmodium sporozoites; (C) Incubation of the drug upon the addition of Plasmodium sporozoites for 2 hours; (D) Incubation of the drug 2 hours from the time of addition of Plasmodium sporozoites up to 2 or 7 days.

FIG. 2—Characterization of HepG2 spheroids during 3D culture, (A) Phase contrast and fluorescence microscopy images of live/dead assay (Live cells, Fluorescein-diacetate; Dead cells, Topro-3) in the first and second weeks of culture (day 4 and 9, respectively). Scale bars. 50 μm. (B) Spheroid diameter in the first and second weeks of culture (Days 4 and 9, respectively). Results are presented as meant±S.D of two independent experiments. (C) Analysis of gene expression levels of 2D cultures and 3D cultures over 15 days of culture for the metabolic genes CYP3A4, CYP2D6 and CYPIA2. Results are presented as mean t SEM of two or three independent experiments.

FIG. 3—Characterization of HepaRG spheroids during 3D culture. (A) Phase contrast and fluorescence microscopy images of live/dead assay (Live cells, Fluorescein-diacetate; Dead cells, Topro-3) during the first and second weeks of culture (Day 4 and 9, respectively). Scale bars: 50 μm. (B) Spheroid diameter in the first and second weeks of culture (Days 4 and 9, respectively). Results are presented as mean±S.D of two independent experiments.

FIG. 4—Optimization of the aggregation of HC-04 cells. Aggregation was induced by culturing the cells for 3 days in medium with 10% to 20% (v/v) FBS, Contrast phase microscopy images representative of HC-04 spheroids from the 2 culture conditions by day 8 of culture. Scale bars: 50 μm.

FIG. 5—Characterization of HC-04 spheroids during 3D culture. (A) Phase contrast and fluorescence microscopy images of live/dead assay (Live cells, Fluorescein-diacetate; Dead cells, Topro-3) in the second week of culture (Day 9), Scale bars: 50 μm. (B) Spheroid diameter in the first and second weeks of culture (Days 4 and 9, respectively). Results are presented as mean±S.D. of three independent experiments. (C) Analysis of gene expression levels of 2D cultures at day 3 and 3D cultures over 15 days of culture for the metabolic genes CYP3A4, CYP1A2 and CYP2D6. Results are presented as mean±SEM of two or three independent experiments.

FIG. 6—Phenotypic characterization of HC-04 spheroids. Detection of; (A) E-cadherin; (8) F-actin; (C) Albumin; (D) Hepatocyte nuclear factor 4 alpha (HNF4α); (E) CYP3a4; (F) CD81. Images from fluorescence microscopy of 10 μm thick cryosections of spheroids from day 9, Scale bars: 50 μm.

FIG. 7—Characterization of PHH spheroids during 3D culture. (A) Phase contrast and fluorescence microscopy images of showing dead cells (Dead cells, Topro-3) at day 3 and 6 of culture, respectively. Scale bars: 100 μm.

FIG. 8—Characterization of of heterotypic spheroid cultures. (A) Phase contrast microscopy image of PHH:HepaRG co-culture at day 3 of culture. Scale bars: 50 μm. (B) Phase contrast microscopy images of HC-04:HepaRG co-culture at day 4 of culture. Scale bars: 50 μm.

FIG. 9—Characterization of P. berghei infection of 3D cultures in dynamic conditions. (A) Phase contrast and fluorescence microscopy images of live/dead assay (Live cells, Fluorescein-diacetate; Dead cells, Topro-3) 48 hours after infection. Scale bars: 100 μm. (B) Infection rate of 3D cultures infected in static and dynamic conditions, expressed as percentage relative to static infection. Luciferase activity was normalized by μg of DNA. Results are the mean±S.D of 4 technical replicates from a single experiment.

FIG. 10—Optimization of HepG2 spheroid culture conditions for infection. Fluorescence microscopy images of viable cells (Fluorescein-diacetate) of HepG2 spheroids after centrifugation at 500, 1000 and 1800×g, the latter with slow acceleration and braking. Scale bars: 100 μm.

FIG. 11—Culture of HepG2 spheroids in 96 well plates. Fluorescence microscopy images of viable cells (Fluorescein-diacetate) of HepG2 spheroids centrifuged at 1800×g for 5 minutes with medium acceleration and brake, and maintained for an additional 48 h in the 98-well plate. Scale bars: 100 μm.

FIG. 12—Optimization of cell-to-sporozoite ratio and mode of contact. (A) Luciferase activity in relative luminescence units (RLU) for centrifuged (black) and non-centrifuged (gray) conditions. Results are the mean of 5 technical replicates±S.D from a single experiment. (B) Luciferase activity of infected HepG2 spheroids relative to the infection of HepG2 2D cultures. Results are the average of at least 3 independent biological experiments±SEM.

FIG. 13—Optimization of cell density at infection. Contrast phase microscopy images of first and second week HepG2 spheroids distribution in a 96-well plate. Scale bars: 100 μm.

FIG. 14—Optimization of Plasmodium infection of HepG2 spheroids. Infection rate of HepG2 spheroids by Pb-Luc (A) and Pb-GFP (B) relative to HepG2 cells cultured in 2D infected at 1:1 cell-to-sporozoite ratio. Results are represented as the mean±SEM of at least 5 independent experiments. (C) Development of Pb-GFP parasites in HepG2 spheroids. Results of GFP intensity normalized to those obtained for HepG2 cells cultured in 2D. Results are presented as mean±EM of at least 5 independent experiments, indicate significant differences by a t-student test (**p<0.01, *p<0.05).

FIG. 15—Plasmodium infection of HC-04 spheroids. Infection rate of Pb-Luc (A) and Pb-GFP (B) for HC-04 spheroids and 2D cell cultures normalized for HepG2. Results represented as the mean±SEM of at least 3 independent experiments. (C) Development of Pb-GFP parasites in HC-04 spheroids. Results of GFP intensity normalized to those obtained for HepG2 cultured in 2D. Results are presented as mean±SEM of at least 3 independent experiments. * indicate significant differences by a t-Student test (*p<0.05).

FIG. 16—Quantification of Plasmodium infection in HC-04 spheroids. (A) Percentage of infected spheroids at 2.5 and 5×10⁴ cell/well in a 1:2 cell-to-sporozoite ratio. Results are mean±S.D of two independent experiments. (B) Number of cells infected per spheroid, infected in 2.5 and 5×10⁴ cell/well in a 1:2 cell-to-sporozoite ratio. Results are mean±S.D of two independent experiments. (C) Fluorescence microscopy image representative of Pb-GFP infection. Scale bar: 100 μm.

FIG. 17—Characterization of Plasmodium development throughout infection. (A) Pb-GFP development over time (24 to 60 h post-infection) in 2D and 3D cultures of HepG2 and HC-04, determined by quantification of GFP intensity. Results are mean±S.D. of three independent experiments (B) Monitoring of sporozoites growth within infected cells at 24 h, 36 h and 48 h post-infection in HC-04 spheroid and 2D cultures. Arrows indicate cells with developing parasites. (C) Detection of the UIS4 paresitophorous vacuole protein at 48 h post-infection using a specific α-UIS-4 antibody. 3D images are projections of 4.2 μm z-stacks. Scale bars; 50 μm.

FIG. 18—In vivo infectivity of merosomes from 3D cultures of HC-04 and 2D cultures of HepG2, determined by quantification of infected red blood cells (RBC). Results are mean±S.D. of one experiment including at least four mice per condition.

FIG. 19—Analysis of drug activity in Pb infected-HC-04 spheroids. Dose-response curve of 3D cultures treated with ATQ. Results are expressed as percentage of infection normalized to the non-treated control. Results are represented as the mean of up to two independent experiments.

FIG. 20—Schematic representation of the preparation of an infected 3D culture according to the present invention and use of the same in a high-throughput screening for anti-malaria drugs.

### EXAMPLES

Unless otherwise specified, all starting materials are obtained from commercial suppliers and used without further purifications. Unless otherwise specified, all temperatures are expressed in ° C. and all reactions are conducted at RT.

### ABBREVIATIONS

ATQ—Atovaquone
DMEM—Dulbecco's Modified Eagle's Medium
DMSO—Dimethyl Suffoxide
ECM—Extracellular matrix
FBS—Fetal bovine serum
F12—Ham's F-12 Nutrient Mixture
GFP—Green fluorescent protein
HNF4α—Hepatocyte nuclear factor 4 alpha
P. berghei—Plasmodium berghei
P. cynomolgi—Plasmodium cynomolgi
P. falciparum—Plasmodium falciparum
P. malariae—Plasmodium malariae
P. ovale—Plasmodium ovale
P. vivax—Plasmodium vivax
Pb-GFP—Plasmodium berghei constitutively expressing GFP
Pb-Luc—Plasmodium berghei, constitutively expressing luciferase
RLU—Relative luminescence units
rpm—Rotations per minute
S.D.—Standard Deviation
STB—Stirred-tank bioreactors
×g—× times gravity

The invention will be illustrated (but not limited), by reference to the specific embodiments described in the following examples.

### 1. Growing and Characterization of the 3D Cultures

### Example 1a: Establishment of 3D Culture of HepG2 Cells

HepG2 spheroids were generated in stirred-tank systems. The culture conditions used for HepG2 spheroids are summarized in Table 1.

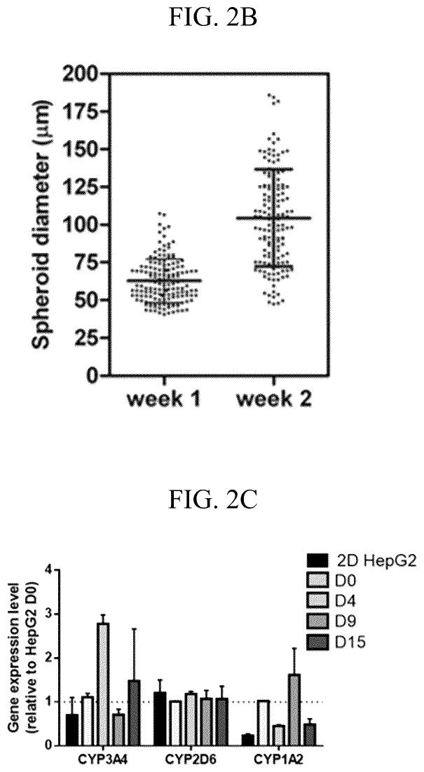

HepG2 cells formed spheroids with high cell viability (FIG. 2). By day 4, HepG2 spheroids were compact, with an average diameter of 63±14 μm (FIG. 2, day 4). Although spheroids presented higher diameter heterogeneity by the second week of culture (FIG. 2, day 9), they are more compact than in the first week, with an average diameter of 104±32 μm (FIG. 2 B). Analysis of basal gene expression of CYP3A4, 2D6 and 1A2 over time indicated that there are no major differences in gene expression over the culture period, showing the metabolism is stable in 3D culture over time (FIG. 2 C).

TABLE 1
Culture conditions for the establishment
of 3D culture of HepG2 cells.

| | |
|---|---|
| Cell inoculum | 0.3 × 10⁶ cell/mL |
| concentration | |
| Culture medium | DMEM + 10% (v/v) FBS + 1% (v/v) PenStrep |
| Agitation rate | 40-100 rpm |
| Feeding regimen | 50% (v/v), every three days |

### Example 1b: Establishment of 3D Culture of HepaRG Cells

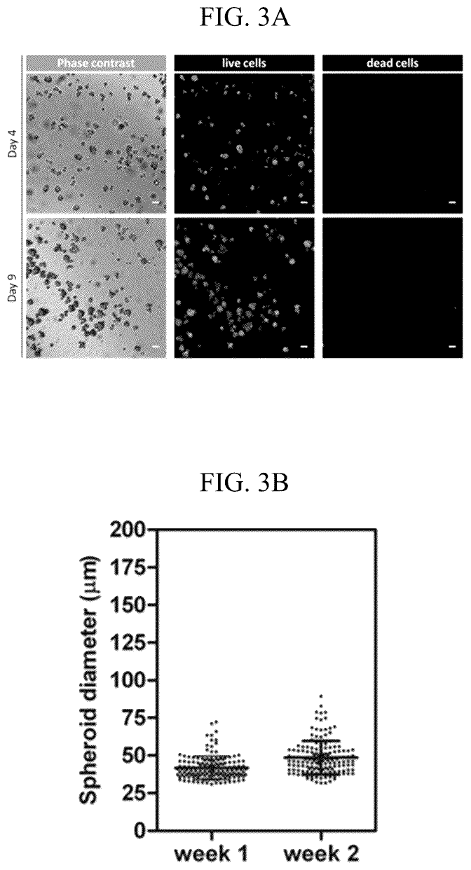

HepaRG spheroids were generated in stirred-tank systems. The optimized 3D culture parameters are summarized in Table 2. Representative images of HepaRG spheroids and spheroid diameter along culture time are shown in FIG. 3. The spheroids had an average diameter of 40±7 μm, and were maintained at least for 2 weeks of culture (FIG. 3 B). The total number of HepaRG cells was maintained throughout the culture time, in contrast to what was observed for HepG2 (data not shown). The differences between 3D cultures of HepG2 and HepaRG cells can be explained by the non-proliferative phenotype of HepaRG cells in 3D, as previously reported by our team [Rebelo, S. P., Costa, R., Estrada, M., et al. (2014) HepaRG microencapsulated spheroids in DMSO-free culture: novel culturing approaches for enhanced xenobiotic and biosynthetic metabolism. Arch Toxicol.], contrary to HepG2 spheroids, which were highly proliferative in 3D culture conditions.

TABLE 2
Culture conditions for the establishment
of 3D culture of HepaRG cells.

| | |
|---|---|
| Cell inoculum | 0.3 × 10⁶ cell/mL |
| concentration | |
| Culture medium | William's E Medium + 2 mM L-Glutamine, 5 μg/ml |
| | Insulin + 10% (v/v) FBS + 1% (v/v) PenStrep |
| Agitation rate | 40-60 rpm |
| Feeding regimen | 20% (v/v), twice a week |

### Example 1c: Establishment of 3D Culture of HC-04 Cells

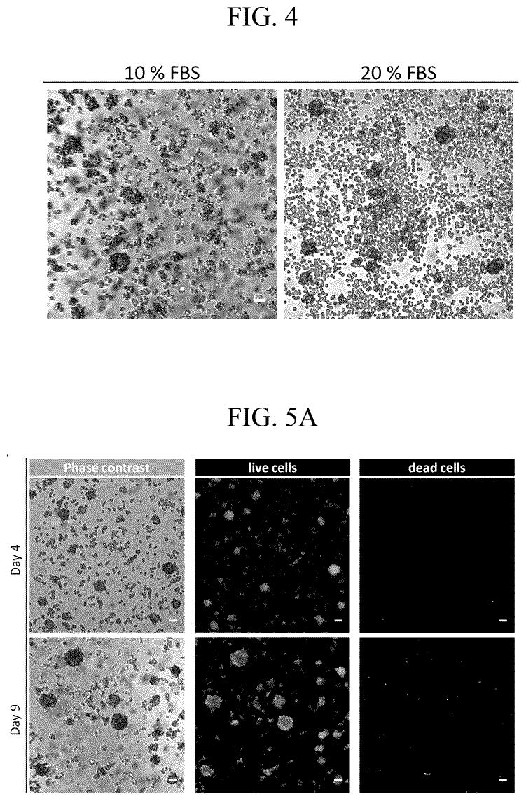

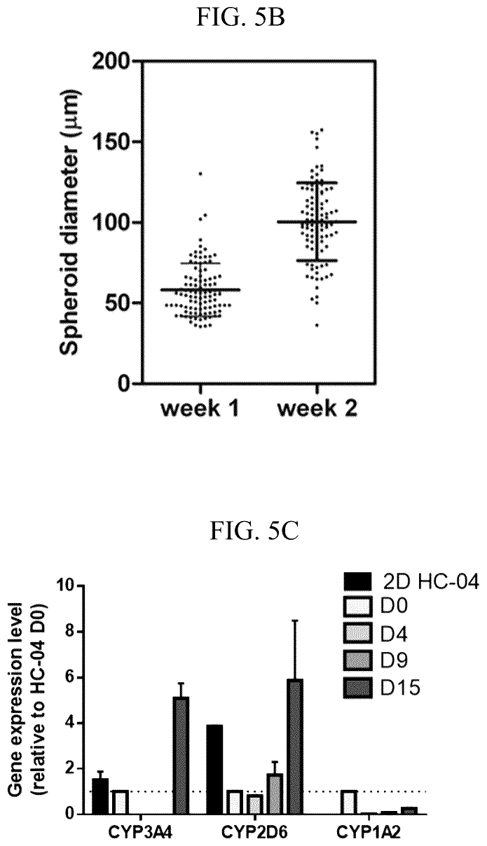

3D cultures of the HC-04 cell line were established based on the conditions implemented for HepG2 cells. When HC-04 cells were cultured in 10% FBS, variation of inoculum concentration and agitation rate had no beneficial effect on cell aggregation efficiency; HC-04 cells formed very few and non-compact spheroids (FIG. 4). Increasing FBS concentration to 20% during the first three days of culture improved aggregation efficiency and generated more compact HC-04 spheroids (FIG. 4). The optimized culture strategy is summarized in Table 3. Using the optimized aggregation strategy, HC-04 cells formed compact spheroids with high cell viability (FIG. 5 A). At day 4 of culture, HC-04 spheroids presented an average diameter of 58±16 μm (FIG. 5 B), which increased throughout culturing time, reaching approximately 100±24 μm by day 9. Analysis of basal gene expression of CYP3A4 and CYP2D6 indicated that, despite the fluctuations in gene expression over the culture period, at the 2^{nd} week of culture (day 15) there was a peak in the expression of these genes. On the other hand, CYP1A2 expression levels decrease over the culture period (FIG. 5 C).

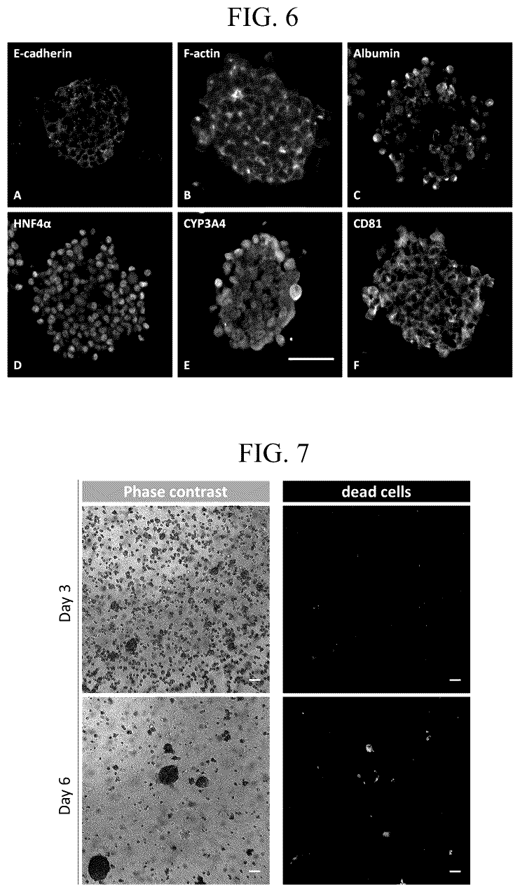

The hepatic phenotype of HC-04 spheroids was characterized by immunofluorescence microscopy (FIG. 6). Detection of E-cadherin in the intercellular junctional spaces indicated tight cell-cell contacts, which were previously reported to be maximized in 3D cultures [Tostões, R, M., Leite, S. B., Serra, M., et al. (2012) Human liver cell spheroids in extended perfusion bioreactor culture for repeated-dose drug testing. Hepatology, 55(4), 1227-1236]. F-actin enrichment in the intercellular regions detected throughout the spheroids indicated high cellular polarization and the presence of bile canaliculi-like structures, typical of hepatic cells. Hepatic identity was further corroborated by detection of albumin, one of the liver-specific biosynthetic products and the presence of the hepatic specific protein HNF4α in all cells of the spheroid. Detection of CY3A4 (FIG. 6 E) confirmed the expression of hepatic metabolizing enzymes by HC-04 cells in spheroids, as well as of CD81, one of the receptors known to be involved in the Plasmodium entry in the hepatocytes [Foquet, L., Hermsen, C. C., Verhoye, L., et al. (2014) Anti-CD81 but not anti-SR-BI blocks Plasmodium falciparum liver infection in a humanized mouse model. Journal of Antimicrobial Chemotherapy, 70(February), 1784-1787], was detected heterogeneously within the spheroid, being mostly accumulated in the cell membranes (FIG. 6 F). Overall, HC-04 cells in spheroids present typical phenotypic hallmarks of hepatocytes.

TABLE 3
Culture conditions for the establishment of 3D cultures of HC-04.

| | |
|---|---|
| Cell inoculum | 0.3 × 10⁶ cell/mL |
| concentration | |
| Culture medium | DMEM F12 + 10% (v/v) FBS + 1% (v/v) PenStrep |
| Agitation rate | 80-105 rpm |
| Feeding regimen | 50% (v/v), every other day |

### Example 1d: Establishment of 3D Culture of Primary Human Hepatocytes

The 3D culture of cryopreserved primary human hepatocytes (PHH) was established based on the previously described strategy for hepatic cell lines, with the same cell inoculum concentration and increasing the initial agitation speed according to Table 4. PHH spheroids were compact after 6 days of culture and the 3D culture was maintained for up to two weeks in stirred-tank vessels (FIG. 7).

TABLE 4
Culture conditions for the establishment
of 3D culture of cryopreserved PHH.

| | |
|---|---|
| Cell inoculum | 0.3 × 10⁶ cell/mL |
| concentration | |
| Culture medium | William's E + 10% (v/v) FBS + hepatocyte |
| | maintenance supplement* |
| Agitation rate | 80-105 rpm |
| Feeding regimen | 25% (v/v), every other day |

*Commercially available, recommended by PHH supplier

### Example 1e: Establishment of Heterotypic Culture of Hepatic Spheroids (3D Co-Culture of HC-04:HepaRG and PHH:HepaRG)

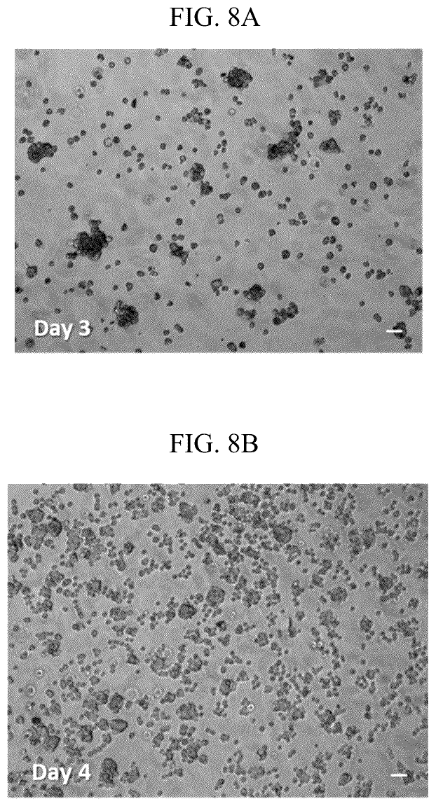

A 3D co-culture of HC-04 and HepaRG cell lines was established based on of the aggregation conditions implemented for HC-04 cells. Cells were co-cultured in a ratio of 2 HC-04:1 HepaRG, in DMEM+F12 culture medium according to Table 3. The co-culture with HepaRG cells had a beneficial effect on cell aggregation, as compared to HC-04 mono-cultures, enabling the generation of spheroids with a FBS concentration of 10% (v/v). Variation of agitation rate from 50 to 80 rpm along two weeks of culture time led to the generation of compact spheroids (FIG. 8 A). Using the optimized aggregation strategy the resulting spheroids presented an average diameter of 65±13 at day 4 of culture, reaching approximately 113±32 μm by day 9 (data not shown).

For the co-culture of PHH with HepaRG cell line, a ratio of 9 PHH:1 HepaRG ratio at 2×10⁵ cell/mL cell density was tested. The aggregation was efficient, with spheroids formed 3 days after inoculation and culture viability was maintained over the culture period (FIG. 8 B). The co-culture strategy with the HepaRG cell line for both cell sources led to an effective aggregation efficiency in the first four days of culture.

### II. Infection of 3D Cultures & Characterization

### Example 2a: Infection of 3D Culture of HepG2 Cells with P. berghei Sporozoites in Dynamic Conditions

For the infection of a large number of spheroids and maintenance in culture for long-term periods, the infection in dynamic conditions using spinner vessels was implemented. Several parameters were considered to establish the dynamic infection, such as the sporozoite and cell concentrations, cell-to-sporozoite ratio and culture volume and agitation during infection, with the aim of maximizing cell-to-sporozoite contact and minimizing the impact of shear stress on the viability of hepatic spheroids. The parameters and conditions used for implementation of infection in dynamic conditions using spinner vessels are summarized in Table 5.

TABLE 5
Culture conditions for the establishment
of infection in dynamic conditions.

| Cell line | | HepG2 |
|---|---|---|
| Culture | Cell concentration (cell/mL) | 0.5 × 10⁶ |
| parameters | | |
| | Volume (ml) | 5 |
| | Agitation speed (rpm) | 40 |
| Infection | Sp concentration (sp/mL) | 0.5 × 10⁶ |
| parameters | | |
| | Cell:sp ratio | 1:1 |

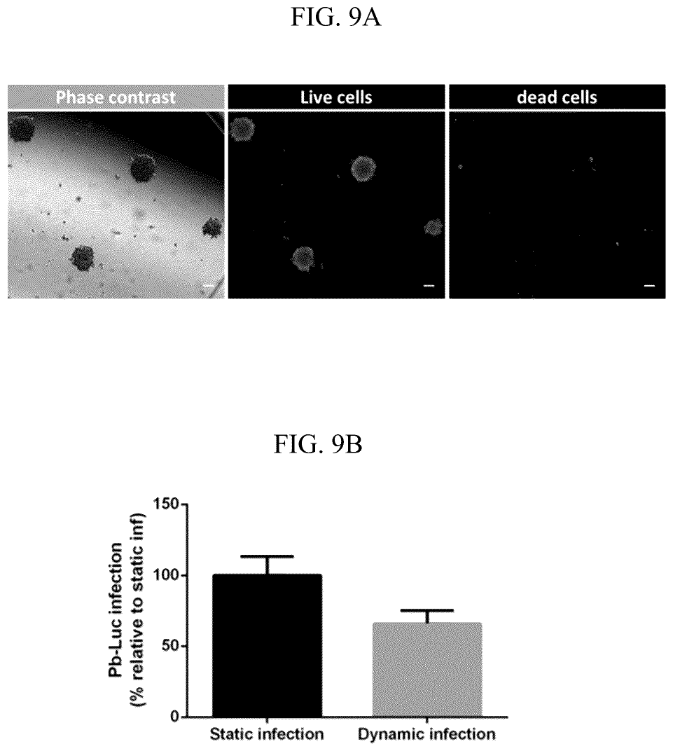

The infection rate in dynamic conditions was assessed in 3D cultures of HepG2 and compared to static conditions using a cell-to-sporozoite ratio of 1:1, at 2.5×10⁴ cell/well. Cell viability 48 h post-infection was high, indicating that the manipulation of culture parameters and resulting shear stress had no impact on spheroid integrity and viability (FIG. 9 A). Moreover, the infection in dynamic conditions was successful (66% comparing with infection in static conditions), indicating that this strategy can be applied for the infection of spheroids. Since the infection in dynamic conditions requires a large quantity of sporozoites, the subsequent examples entailing infection of other hepatic cell sources and characterization of infection were performed in static conditions.

### Example 2b: Infection of 30 Culture of HepG2 Cells with P. Berghei Sporozoites in Static Conditions

Infection parameters including cell concentration, cell-to-sporozoite ratio, cell-to-sporozoite mode of contact and culture time of the spheroids were optimized. Sporozoites were obtained from the dissection of the salivary glands of infected Anopheles stephensi mosquitoes. Following mechanical disruption of salivary glands, the sporozoite suspension was kept on ice for up to 3 hours, until sporozoites were employed to inoculate the cells in culture.

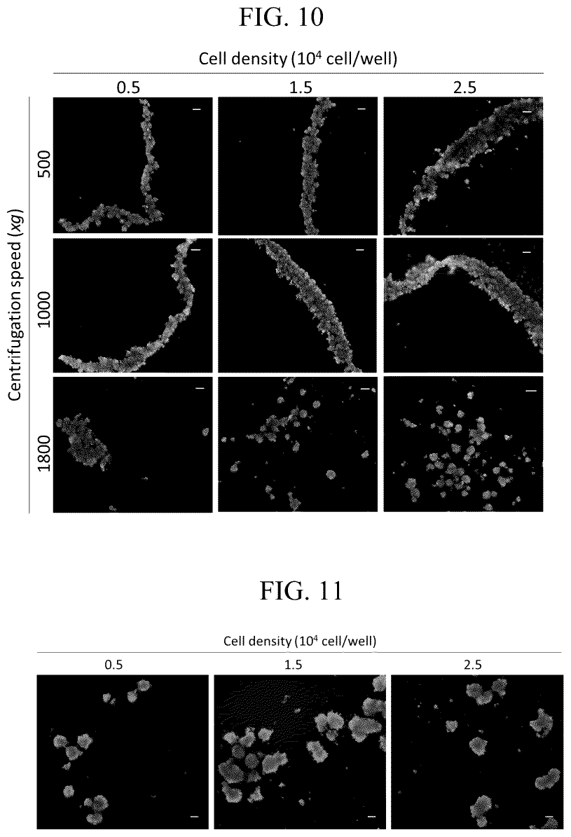

For the implementation of standard infection conditions in spheroids, the effect of centrifugation and subsequent static culture in 96-well plates was assessed. All centrifugation speeds led to spheroid fusion except in the condition where the centrifugation speed was gradually reached and gradually decreased (equivalent to acceleration and braking profiles 5 in a Rotina420R, Hettich centrifuge), FIG. 10. Under this condition, spheroids retained integrity, without fusion, and maintained high cell viability independently of cell concentration, in the central rows of the plate (rows C, D and E). Thus, culture progression was evaluated for 48 hours after centrifugation using the setting described. Readouts were cell viability and spheroid fusion (FIG. 11). After 48 hours in culture, HepG2 spheroids maintained high cell viability with minimal spheroid fusion in the three cell concentrations tested.

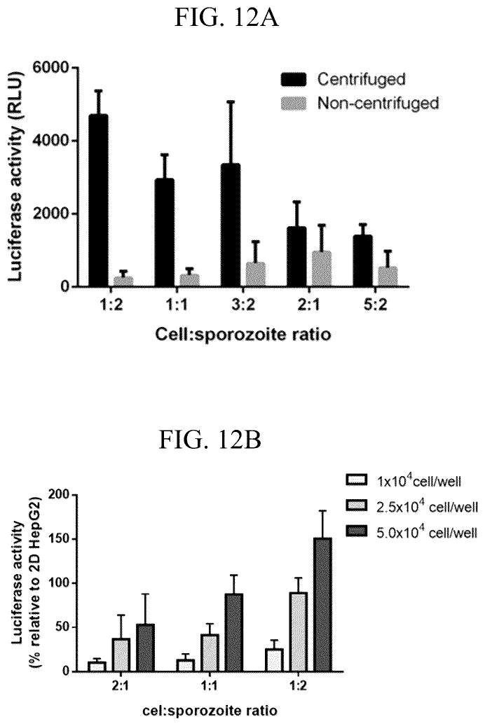

Initially, a preliminary assay employing reporter lines of Plasmodium berghei, constitutively expressing luciferase (Pb-Luc) or GFP (Pb-GFP), was performed to optimize the range of cell-to-sporozoite ratios and mode of contact. Pb-Luc parasites enable measuring infection by luminescence readings of cell lysates following addition of the luciferin substrate. Pb-GFP parasites enable measuring infection flow cytometry analysis. Such analyses allow measuring the percentage of invaded cells (% GFP-positive cells) and the development of the parasite inside the hepatic cells (GFP intensity). The conditions tested and readouts employed are depicted in Table 6 and the results obtained are presented in FIG. 12.

TABLE 6
Parameters tested for optimization of infection of HepG2
spheroids with Pb-Luc and the correspondent readout.

| Conditions Tested | Readout |
|---|---|
| 2D vs 3D | Infection |
| 2^{nd} week spheroids Cell densities-0.5, 1, 1.5, 2 and | rate:Luciferase |
| 2.5 × 10⁴ cell/well | activity |
| Cell:sporozoite ratio-1:2, 1:1, 3:2, 2:1 and 5:2 | (luminescence- |
| Centrifuged (1800 xg) vs. non-centrifuged | based assay) |

HepG2 spheroids presented higher infection rate when cell-to-sporozoite contact was promoted by centrifugation (FIG. 12 A). In these conditions, the highest infection rates were obtained for cell-to-sporozoite ratios of 1:2 and 1:1, using the cell concentrations of 2.5 and 5×10⁴ cell/well (FIG. 12 B).

Therefore, the preferred procedure for infection was: (i) Distribution of spheroids from spinner vessel to 96 well plates for infection; (ii) Promotion of sporozoite-to-cell contact by centrifugation at 1800×g for 5 min with medium acceleration and braking; (iii) Maintenance of spheroids in 96-well plates, in static conditions, for 48 hours post-infection, for infection assessment.

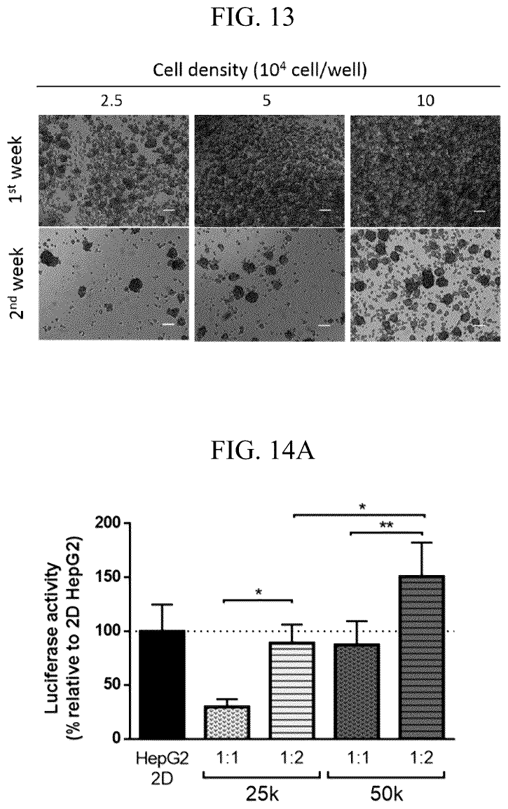

Cell-to-sporozoite ratios of 1:2 and 1:1 were selected to proceed with the optimization of P. berghei infection. Aiming to maximize cell-to-sporozoite contact, cell density at infection was optimized to achieve the maximum coverage of the well surface. The results are presented in FIG. 13. Inoculation of 2.5×10⁴ and 5×10⁴ cell/well (7.8×10⁴ and 15.6×10⁴ cell/cm², respectively) led to 60-80% of the well surface covered by spheroids, with no spheroid fusion being observed after 48 hours in culture. Conversely, when using a seeding density of 10×10⁴ cell/well, spheroid fusion occurred. Thus, the lower cell concentrations (2.5×10⁴ and 5×10⁴ cell/well) were selected for further optimization of HepG2 spheroids infection with P. berghei, by evaluating two cell-to-sporozoite ratios (1:1 and 1:2) and using spheroids generated by 1 or 2 weeks in culture.

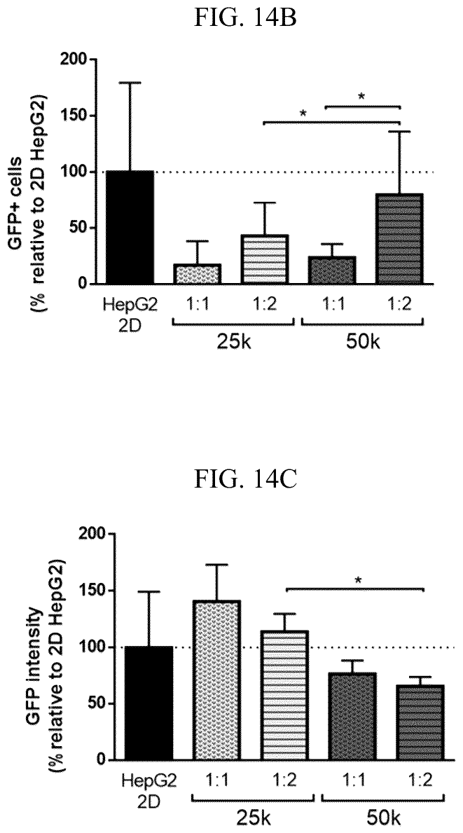

The results showed that higher infection rates were obtained with hepatic spheroids generated by two weeks in culture (Table 6). Moreover, the infection rates could be maximized using 5×10⁴ cell/well and a 1:2 cell-to-sporozoite ratio for both lines of P. berghei (150% and 80% relative to HepG2 2D cells, for Pb-Luc and Pb-GFP respectively; FIG. 14 A; B). Nonetheless, 2.5×10⁴ cell/well in 1:2 cell-to-sporozoite ratio may be considered as alternative in case of limited sporozoite availability, leading to a Pb-Luc infection rate of 89% relative to 20 cultures (FIG. 14 A; B). In general, the infection rate of HepG2 spheroids is comparable to the obtained for 2D cells (FIG. 14 A; B).

TABLE 6
Spheroids in 1^{st} week of culture vs 2^{nd} week of culture. Infection
rate represented as luciferase activity normalized to that
of HepG2 2D cultures infected in 1:1 cell-to-sporozoite ratio. Results
from at least two independent experiments, except for 5 ×
10⁴ cell/well in 1:2 cell-to-sporozoite ratio.

| Cell:Pb | Cell density | Infection rate (% to 2D) | |
|---|---|---|---|
| ratio | (10⁴ cell/well) | Spheroids 1^{st} week | Spheroids 2^{nd} week |
| 1:1 | 2.5 | 15.2 ± 10.9 | 21.9 ± 8.5 |
| 1:2 | | 26.1 ± 13.8 | 78.9 ± 23.5 |
| 1:1 | 5 | 46.4 ± 0.9 | 63.6 ± 51.6 |
| 1:2 | | 43.6 | 140.5 ± 82.7 |

Both analytical methods, assessment of luciferase activity or GFP fluorescence, were consistent in identifying the infection conditions leading to the highest infection rates (FIG. 14 A; B). The differences in the infection rates observed might be explained by the differences inherent to the two analytical methods employed. For Pb-GFP, infection rate reflects the number of infected cells (percentage of GFP-positive cells) or the development of the parasite (GFP intensity). Conversely, infection with Pb-Luc is analyzed by luciferase activity, and infection rates cumulatively reflect the number of infected cells as well as the development of the Pb-Luc parasite.

Sporozoites were able to develop in HepG2 spheroids, presenting, in all the conditions employed, a development above 65% of that observed in 2D cultures (FIG. 14 C) and higher for the lower cell density conditions (2.5×10⁴ cell/well).

Given the data obtained, an optimal strategy for P. berghei infection of HepG2 spheroids was implemented using: (i) spheroids from two-week cultures; (ii) a cell density of 5×10⁴ cell/well; and (iii) a 1:2 cell-to-sporozoite ratio.

### Example 2c: Infection of 30 Culture of HC-04 CElls with P. berghei Sporozoites in Static Conditions

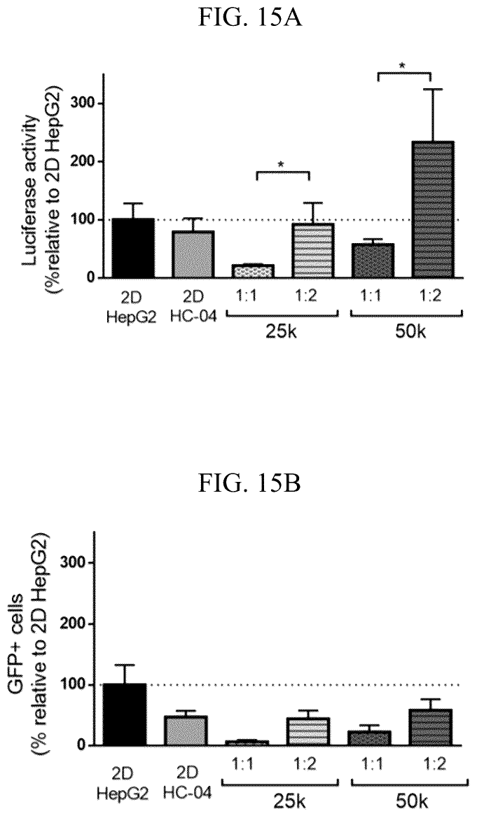

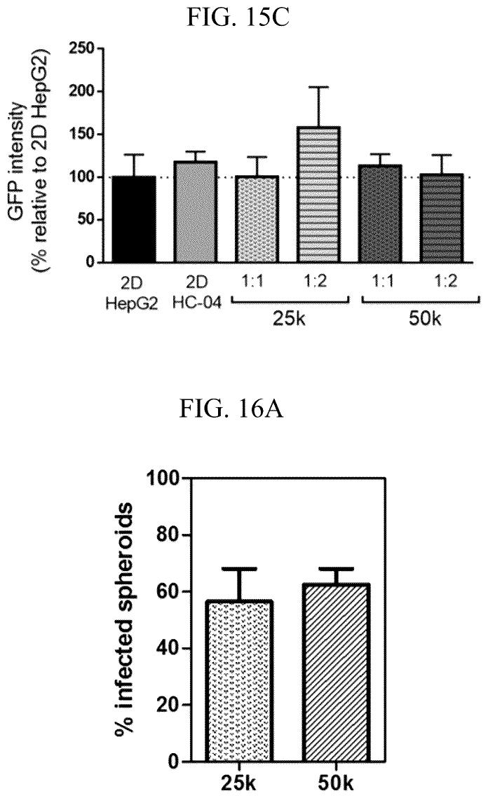

HC-04 cells were infected by both P. berghei parasite lines. In 2D cultures, the infection rate of HC-04 cells was approximately 79% and 47% of the one observed for HepG2 cells under 2D conditions for Pb-Luc and Pb-GFP, respectively (FIG. 15 A; B). Like for HepG2 cells, P. berghei infection was optimized in HC-04 spheroids by evaluating different (i) cell-to-sporozoite ratios and (ii) cell densities. The infection rate could be maximized using a cell-to-sporozoite ratio of 1:2 and a cell density of 5×10⁴ cell/well (FIG. 15 A; B), similarly to what was described above for HepG2 (FIG. 14 A; B). For all conditions tested, Pb-GFP development in 3D cultures of HC-04 was comparable or higher than in 2D cultures of HepG2 (FIG. 15 C).

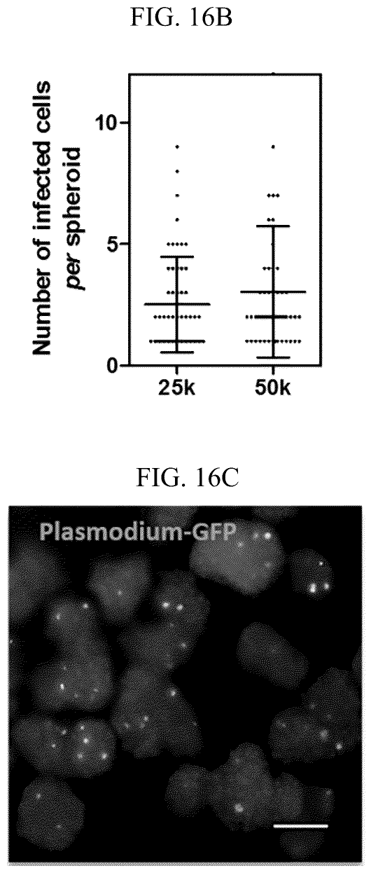

The percentage of spheroids infected by Pb-GFP at 1:2 of cell-to-sporozoite ratio was quantified by fluorescence microscopy, for cell densities of 2.5×10⁴ and 5×10⁴ cell/well. In both conditions more than 55% of the spheroids were infected (FIG. 16 A), with an average of approximately 3 infected cells per infected spheroid (FIG. 16 B).

### Example 2d: Assessment of Parasite Development Over Time in 30 Cultures

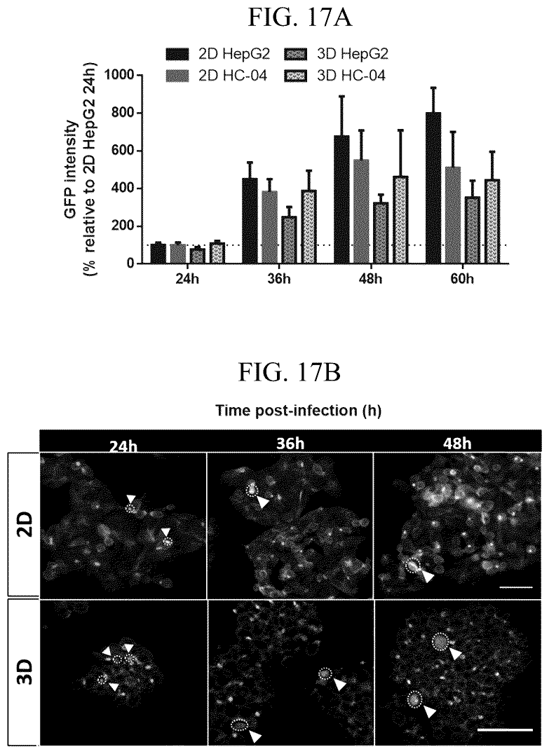

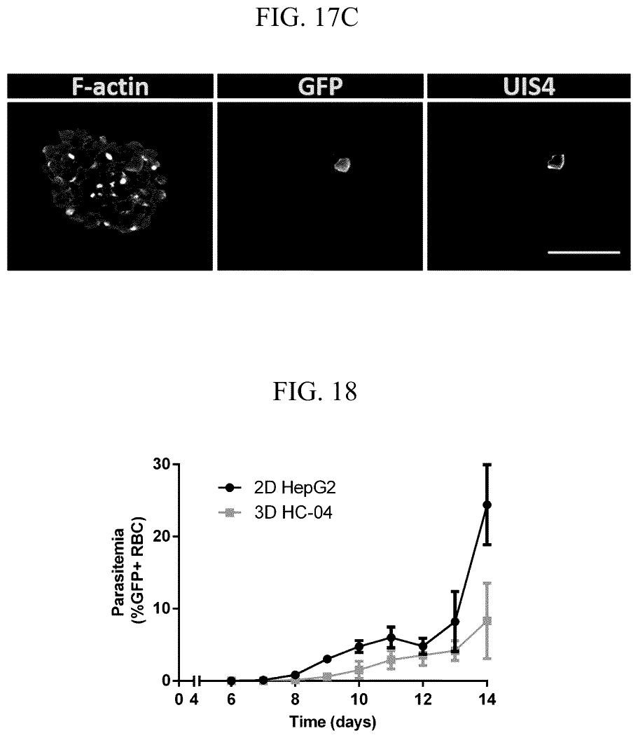

In addition to the implementation and optimization of P. berghei infection in 30, the characterization of parasite development was performed for both hepatic cell lines (HepG2 and HC-04). Parasite development observed 60 hours post-infection was characterized by quantification of GFP intensity. A comparable profile of development was observed for all the conditions tested (20 and 3D; HepG2 and HC-04) (FIG. 17 A), P. berghei development dynamics over showed an increasing profile reaching its maximum value at 48 h, after which is maintained up to 60 h post-infection (FIG. 17 A). Concomitantly, Pb-GFP was able to replicate inside hepatic cells that have been effectively invaded, leading to an increase in the size of the infected hepatic cells both in 2D and 3D (FIG. 17 B). Moreover, the detection of UIS4 at 48 hours post-infection, a protein in the parasitophorous vacuole membrane, confirmed the parasite development inside the parasitophorous vacuole (FIG. 17 C). To assess whether the parasite development in 3D cultures is complete, the release of merosomes was evaluated and in both 2D and 3D cultures from both cell lines (HepG2 and HC-04) there was detection of merosomes in the culture supernatant at 72 h post-infection. The culture supernatant containing merosomes was injected into mice and parasitemia was monitored over time by evaluating the percentage of infected red blood cells (RBC) (FIG. 18). Parasitemia was detected in mice for all the conditions tested, showing that the sporozoite development in 2D and 3D cultures is comparable and results in mature merosomes containing infective merozoites.

### Example 2e: Infection of 3D Co-Culture of HC-04 Cells and HepaRG with P. Berghei Sporozoites

The characterization of HC-04 metabolic activity and its suitability to be used as an in vitro model for drug screening is scarce. This may represent a major limitation for anti-Plasmodium drug assessment in this model, given the importance of liver metabolic activity for the correct metabolization of some anti-Plasmodium drugs (e.g, primaquine). In order to overcome this limitation, strategies based on co-culture systems were considered. Here, HepaRG cell line was selected to pursue a co-culture strategy, since these cells have been previously described as a more accurate surrogate of liver function among the available human hepatic cell lines platforms [Rebelo, S. P., Costa, R., Estrada, M., et al. (2014) HepaRG microencapsulated spheroids in DMSO-free culture: novel culturing approaches for enhanced xenobiotic and biosynthetic metabolism. Arch Toxicol.]. Moreover, previous reports have shown that co-cultures of primary hepatocytes and HepaRG could extend hepatocyte integrity and fitness, as well as improve P. cynomolgi infection [Dembéié, L., Franetich, J., Lorthiois, A., et al, (2014) Persistence and activation of malaria hypnozoites in long-term primary hepatocyte cultures. Nature Medicine, 20(3), 307-312].

It was assessed whether the co-culture of HC-04 and HepaRG would have an impact on P. berghei infection. A 3:1 ratio of HC-04 to HepaRG cells was tested. Infection of co-cultures and HC-04 monocultures were performed with Pb-GFP in the optimized conditions described above (two weeks spheroids, cell density 2.5×10⁴ and 5×10⁴ cell/well in a 1:2 ratio). The results are presented in Table 7.

TABLE 7
Plasmodium infection of HC-04:HepaRG spheroids. Infection
by Pb-GFP represented as the frequency of GFP-positive
cells. Data from a single experiment.

| | | |
|---|---|---|
| Cell:Pb | Cell density | Infection rate (% of GFP+ cells) |

| ratio | (10⁴ cell/well) | Monoculture | Co-culture |
|---|---|---|---|
| 1:2 | 2.5 | 0.56 | 0.19 |
| | 5 | 0.37 | 0.40 |

The results indicate that co-culture did not influence the infection rate in the best condition identified for infection of HC-04 spheroids (1:2 cell-to-sporozoite ratio and cell density of 5×10⁴ cell/well). Thus, this co-culture strategy constitutes a promising alternative to improve the metabolic capacity of the system, as compared to HC-04 monocultures.

### III. In Vitro Testing of Reference Drugs Against Infected of 3D Cultures

### Example 3: Test of Reference Anti-Plasmodium Drugs Primaquine and Atovaquone

The suitability of the platform presented in this invention for drug screening purposes of anti-infective agents was explored using one reference drug. Atovaquone (ATQ), requiring no metabolization to target the liver-stage Plasmodium infection.

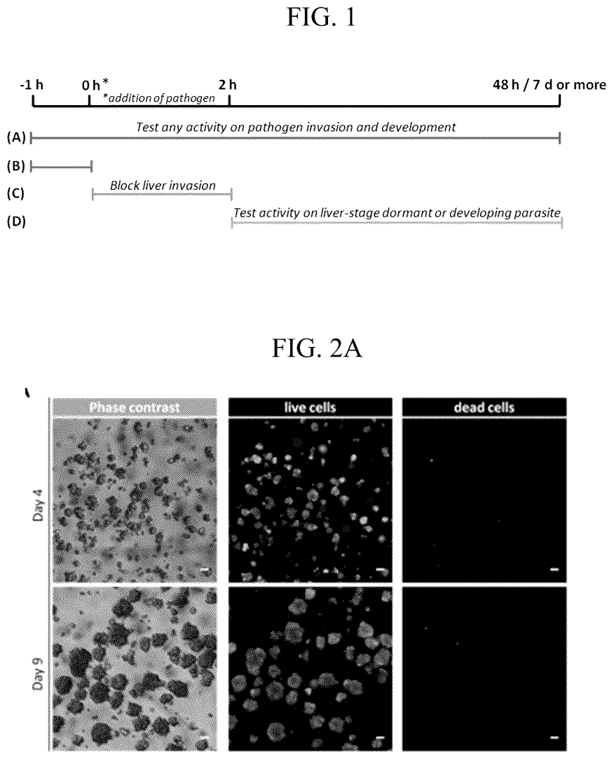

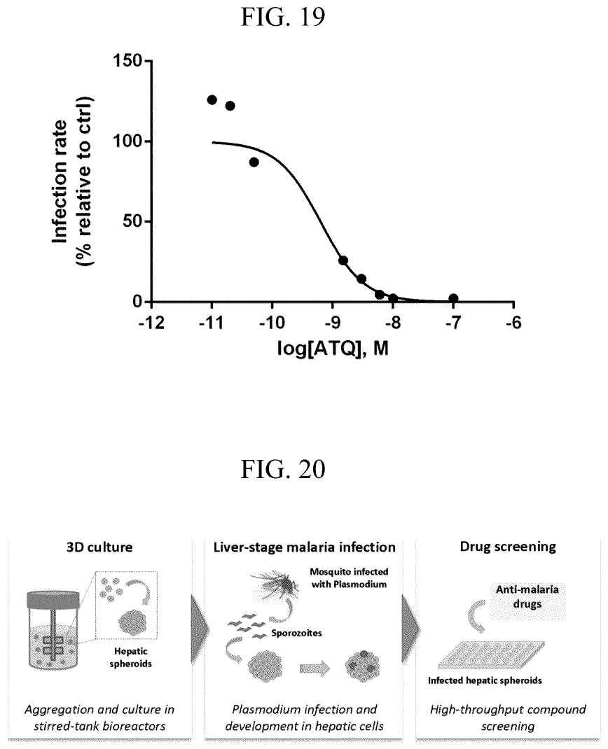

HC-04 3D cultures were infected with Pb-Luc in the optimized conditions described above (cell density of 2.5×10⁴ cell/well in a 1:2 ratio). The assessment of drug effect in the infection was performed by incubating the drug at a range of concentrations from 0.01 to 100 nM for 1 hour before incubation with the sporozoites and the readout was performed 48 hours after sporozoites addition, described as incubation regimen (A) in the detailed description of the invention section (FIG. 1). The drug concentrations employed were shown not to affect cell viability. A dose response curve was established for the 3D cultures treated with ATQ and a 0.6 nM half inhibitory concentration (IC₅₀) for sporozoite infection was determined. The highest concentrations tested led to a decrease of more than 90% of infection (FIG. 19). ATQ performed similarly in 2D and in 3D cultures (data not shown).

1-18: (canceled)

19: A method for the production of a 3D cell culture containing hepatic cells, said method comprising:
(a) inoculating a single-cell suspension, containing hepatic cells expanded in 2D culture, in an agitation-based culture system;
(b) agitating the resulting cell culture at an agitation rate of 40 to 110 rpm; and/or
(c) incubating the resulting 3D cell culture containing cell aggregates with a pathogen, wherein the cell aggregates are spheroids with an average diameter in the range of 50 μm to 200 μm.

20: The method for the production of a 3D cell culture according to claim 19,
wherein the incubation is performed under static conditions, wherein the 3D cell culture containing the cell aggregates, together with the pathogen, is exposed to centrifugation at up to 1800×g, or
wherein the incubation is performed under dynamic conditions, wherein the cell culture volume is reduced, and the 3D cell culture is exposed to agitation.

21: A 3D cell culture containing hepatic cells obtainable with the method according to claim 19.

22: A screening method, comprising:
(a) incubating a 3D cell culture containing hepatic cells with a compound; and
(b) monitoring of pathogen invasion, compound clearance and/or development of host cells;
wherein the 3D cell culture comprises
a mixture of a synthetic cell culture medium and cell aggregates consisting of hepatic cells in a stirred-tank bioreactor or a spinner vessel, wherein the hepatic cells are HepG2 and/or HC-04 cells, wherein the hepatic cells are infected by a pathogen, the pathogen being Plasmodium berghei or Plasmodium vivax, and wherein the cell aggregates are spheroids with an average diameter in the range of 50 μm to 200 μm.

23: A method, comprising:
contacting a compound with a 3D cell culture, and
determining a cytotoxic effect and/or metabolic properties of the compound contacted with the 3D cell culture and/or an effect of the compound contacted with the 3D cell culture on the pathogen;
wherein the 3D cell culture comprises
a mixture of a synthetic cell culture medium and cell aggregates consisting of hepatic cells in a stirred-tank bioreactor or a spinner vessel, wherein the hepatic cells are HepG2 and/or HC-04 cells, wherein the hepatic cells are infected by a pathogen, the pathogen being Plasmodium berghei or Plasmodium vivax, and wherein the cell aggregates are spheroids with an average diameter in the range of 50 μm to 200 μm.

24: The method according to claim 19, wherein the 3D cell culture is a mono-culture or a co-culture.

25: The method according to claim 19, wherein the pathogen is a reporter strain.

26: The method according to claim 19, further comprising a soluble extracellular matrix.

27: The method according to claim 26, wherein the soluble extracellular matrix comprises at least one material selected from the group consisting of laminin, fibronectin, collagen and a biocompatible biomaterial.

28: The method according to claim 19, wherein the 3D cell culture comprises
a mixture of a synthetic cell culture medium and cell aggregates consisting of hepatic cells in a stirred-tank bioreactor or a spinner vessel, wherein the hepatic cells are HepG2 and/or HC-04 cells, wherein the hepatic cells are infected by a pathogen, the pathogen being Plasmodium berghei or Plasmodium vivax, and wherein the cell aggregates are spheroids with an average diameter in the range of 50 μm to 200 μm.
